(19) United States
(12) Patent Application Publication
(10) Pub. No.: US 2022/0298471 A1
(43) Pub. Date: Sep. 22, 2022

(54) METHOD AND DEVICE FOR FORMING A GEL PARTICLE SLURRY

(71) Applicant: CASE WESTERN RESERVE UNIVERSITY, Cleveland, OH (US)

(72) Inventors: Eben Alsberg, Chicago, IL (US); Oju Jeon, Broadview Heights, OH (US)

(21) Appl. No.: 17/698,539

(22) Filed: Mar. 18, 2022

Related U.S. Application Data
(60) Provisional application No. 63/162,846, filed on Mar. 18, 2021.

(51) Int. Cl. C12N 5/00; B01J 2/10

(52) U.S. Cl. CPC C12N 5/0018; B01J 2/10; C12N 2513/00; C12N 2533/74; C12N 2537/10

(57) ABSTRACT

A method of forming a gel particle slurry includes providing a first solution that includes a cross-linkable hydrogel polymer macromer and an optional first crosslinker in a first depot and optionally a second solution in a second depot that is separated from the first depot by a mixing unit that includes a mixing element; and reversibly transferring the first solution and the optional second solution through the mixing unit between the first depot and the second depot such that the first solution and the optional second solution are mixed and agitated to form the gel particle slurry.

### RELATED APPLICATION

This application claims priority from U.S. Provisional Application No. 63/162,846, filed Mar. 18, 2021, the subject matter of which is incorporated herein by reference in its entirety.

### GOVERNMENT FUNDING

This invention was made with government support under AR063194, AR066193, and AR069564 awarded by the National Institutes of Health. The government has certain rights in the invention.

### BACKGROUND

Over the past decades, scaffolding approaches have been widely used to create functional tissues or organs in tissue engineering and regenerative medicine fields. However, the use of biomaterial-based scaffolds faces several challenges, such as interference with cell-cell interactions, potential immunogenicity of the materials and their degradation byproducts, unsynchronized rates of scaffold degradation with that of new tissue formation, and inhomogeneity and low density of seeded cells. To overcome these limitations of scaffold-based approaches, scaffold-free tissue engineering has recently emerged as a powerful strategy for constructing tissues using multicellular building blocks that self-assemble into geometries such as aggregates, sheets, strands and rings. These building blocks have been organized and fused into larger and more complicated structures, sometimes comprised of multiple cell types, and then they produce extracellular matrix (ECM) to form mechanically functional three-dimensional (3D) tissue constructs. However, it is still difficult to precisely control the architecture and organization of cell-only condensations to mimic sophisticated 3D structures of natural tissues and their structure-derived functions.

Recently, 3D printing has been applied in tissue engineering with the potential to create complicated 3D structures with high resolution using cell-free or cell-laden bioinks. Digital imaging data, obtained from computed tomography scans and magnetic resonance imaging, provide instruction for the desired geometry of printed constructs. Biodegradable thermoplastics, such as polycaprolactone, polylactic acid, and poly(lactic-co-glycolic acid), are advantageous for printing as stable constructs with delicate structural control can be formed due to the mechanical integrity of original materials.

### SUMMARY

Embodiments described herein relate to a method of forming a gel particle slurry that can be used, for example, as a support medium for three dimensional (3D) bioprinting. The method includes providing a first solution that includes a cross-linkable hydrogel polymer macromer and an optional first crosslinker in a first depot and optionally a second solution in a second depot that is separated from the first depot by a mixing unit that includes a mixing element. The first solution and the optional second solution are reversibly transferred through the mixing unit between the first depot and the second depot such that the first solution and the optional second solution are mixed and agitated by the mixing element and the cross-linkable polymer macromer forms a gel particle slurry.

### BRIEF DESCRIPTION OF DRAWINGS

FIG. 1 illustrates a schematic of mixing unit in accordance with an embodiment described herein.

FIGS. 2(A-B) illustrate images of (A) custom-made female-female luer lock mixing unit and (B) two syringes connected with the mixing unit.

FIG. 3 illustrates microphotographs of OA and OMA microgels made with the mixing unit. Scale bars indicate 500 μm.

FIG. 4 illustrates plots and graphs showing rheological properties of the alginate slurries.

FIG. 5 illustrates plots showing self-healing property of the alginate slurries.

FIG. 6 illustrates images and graphs 3D printed hMSC filaments with various needles into the 5OX20MA OMA slurry.

FIG. 7 illustrates 3D printed hMSC-only structures with hMSC into the 5OX20MA OMA slurry.

FIG. 8 illustrates 3D printed ears with various OMA slurries as a bioink.

### DETAILED DESCRIPTION

Embodiments described herein relate to a method of forming a gel particle slurry that can be used, for example, as a support medium for three dimensional (3D) bioprinting. The method includes providing a first solution that includes a cross-linkable hydrogel polymer macromer and an optional first crosslinker in a first depot and optionally a second solution in a second depot that is separated from the first depot by a mixing unit that includes a mixing element. The first solution and the optional second solution are reversibly transferred through the mixing unit between the first depot and the second depot such that the first solution and the optional second solution are mixed and agitated by the mixing element and the cross-linkable polymer macromer forms a gel particle slurry.

Advantageously, the method provides fabrication of a gel particle slurry with minimal equipment and under sterile conditions without the need for preservatives (e.g., 70% ethanol) for long-term storage. Moreover, a washing process, which can potentially cause degradation of the gel particle slurry, is not needed to remove preservatives. Additionally, alginate with high degree of oxidation can be used for making the gel particle slurry because alginate slurries formed with higher than 10% oxidation degree using a grinding method can degrade during a washing process.

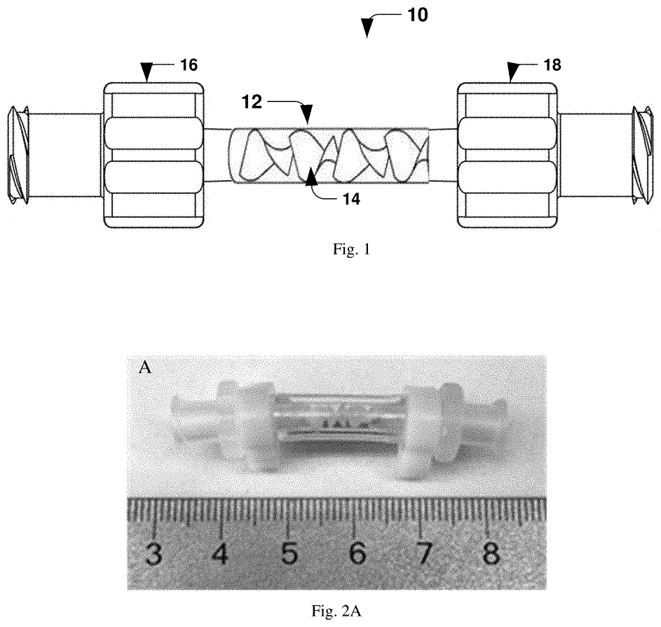

FIG. 1 illustrates a schematic of a mixing unit 10 in accordance with an embodiment described herein. The mixing unit includes a mixing chamber 12 and mixing element 14 that is contained within the chamber 12. The mixing chamber 12 is in fluid communication with and extends between a first luer lock 16 and a second luer lock 18. The first luer lock 16 and the second luer lock 18 are connected respectively to the first end and the second end of the mixing chamber 12.

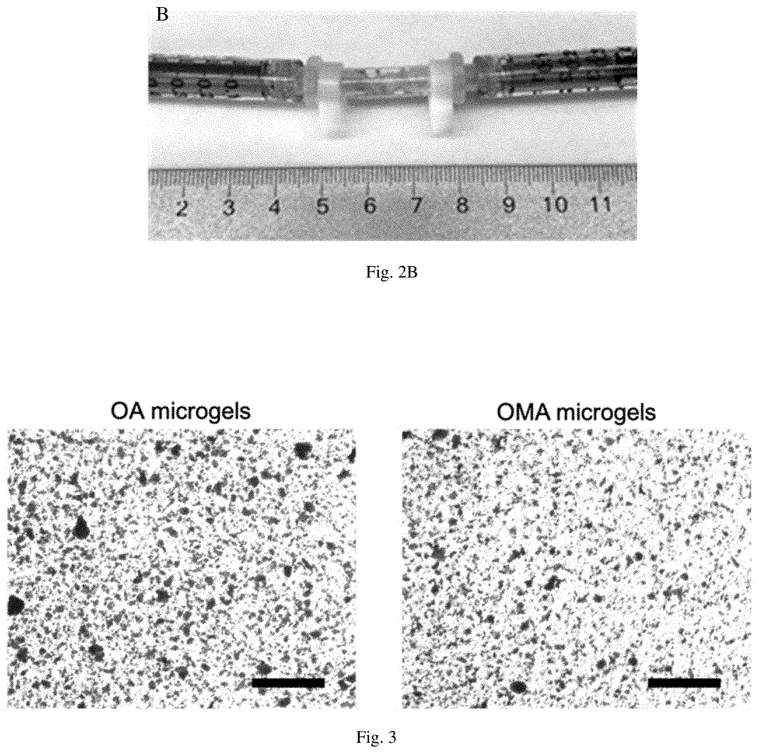

As illustrated in FIG. 2A, the first luer lock 16 and second luer lock 18 are configured to receive and provide a leak free-connection with a first depot, such as a first syringe, and a second depot, such as a second syringe. A first solution that includes a cross-linkable hydrogel polymer macromer and an optional first crosslinker is provided in the first depot and optionally a second solution is provided in a second depot for reversible transfer through the mixing unit between the first depot and the second depot.

The mixing chamber 12 includes a tube or substantially cylinder-shaped wall that defines a volume in which the mixing element 14 is confined and through which the first solution and optional second solution is reversibly transferred between the first depot and the second depot. The mixing element 14 can include a static screw or helical mixing element that extends within the chamber 12 substantially the length of the chamber 12 and provides a continuous in-line mixing of the first solution and the optional second solution as they are passed back and forth through the mixing chamber to form the gel particle slurry.

In some embodiments, the static screw or helical mixing element 14 can include alternating helical elements, each set 90° to an adjacent element for thorough blending or mixing of the first solution and optional second solution. A first helical element of the static helical mixing element 14 rotates the flow of the first solution and the optional second solution in one direction, then the direction is reversed at the next element creating a further mixing effect. The flow of the mixed solutions is forced to be inverted completely so that the stream is continuously moved from the center of the mixing element 14 to an inner chamber wall and hack again.

In some embodiments, the first solution and the optional second solution is reversibly transferred through the mixing unit 10 between the first depot and the second depot such that the first solution and the optional second solution are mixed and agitated to form the gel particle slurry. The first solution and the optional second solution can be reversibly transferred back and forth through the mixing unit at least 10 times, 20 times, 30 times, 40 times, 50 times, 60 times, 70 times, 80 times, 90 times or more to form the gel particle slurry. The gel particle slurry can optionally be further mixed back and forth at least 10 times, 20 times, 30 times, 40 times, 50 times, 60 times, 70 times, 80 times, 90 times at least every 10 minutes, 20 minutes, 30 minutes, 40 minutes, 50 minutes, 60 minutes or more prior to ejection into a printing dish or a cell culture plate.

In some embodiments, the gel particle slurry formed by reversibly transferring the first solution and optional second solution through the mixing unit 10 can include hydrogel particles having an average diameter of about 5 nm to about 10 mm, for example, about 100 nm to about 1000 μm, about 1 μm to about 500 μm, about 25 μm to about 400 μm, or about 50 μm to 200 μm. The hydrogel particles can have substantially homogenous or similar diameters or include particles of varying diameters to provide a heterogenous mixture of the hydrogel particles.

In some embodiments, the first solution provided in the first depot includes a cross-linkable hydrogel polymer macromer and an optional first crosslinker. Optionally, the second solution can include a second crosslinker and/or polymer macromer that is capable of crosslinking the cross-linkable hydrogel polymer macromer.

In some embodiments, the first cross-linker is different than the second crosslinker.

In some embodiments, the cross-linkable hydrogel polymer macromers are at least partially crosslinked.

In some embodiments, the cross-linkable hydrogel polymer macromers include a plurality of acrylated and/or methacrylated polymer macromers. For example, the acrylated and/or methacrylated, polymer macromers are polysaccharides, which are optionally oxidized.

In some embodiments, the first crosslinker can be an ionic crosslinker and/or a photoinitiator, and the second crosslinker can be an ionic crosslinker and/or photoinitiator.

In some embodiments, the cross-linkable hydrogel polymer macromer include oxidized, acrylated and/or methacrylated alginates.

In some embodiments the first depot is a first syringe and the second depot is a second syringe. The first syringe can include an aqueous solution of oxidized acrylated and/or methacrylated alginate and an optional photoinitiator and the second syringe includes a calcium sulfate slurry.

In some embodiments, the gel particle slurry is self-healing, shear thinning, cross-linkable and/or biocompatible.

In other embodiments, the get particle slurry can be used to form a gel particle support medium by transferring the gel particle slurry to a container.

In some embodiments, the gel particle support medium can be a self-healing, shear thinning, crosslinkable, biocompatible hydrogel support medium that can maintain a printed bioink in a defined shape during printing of the bioink and optionally during culturing of cells of the bioink. The hydrogel support medium can behave as a viscous fluid during printing and be resistant to flow before and after printing. For example, initially, the hydrogel support medium is in a flow-resistant or solid-like state before being printed with the bioink. The hydrogel support medium becomes fluidized under the increased shear stress caused by printing the bioink into the hydrogel support medium. Then, after the printing is finished and the increased shear stress is removed, the hydrogel support medium can self-heal and form a flow-resistant or solid-like stable support medium. The hydrogel support medium can be further crosslinked after printing to maintain the defined shape of the printed first bioink during culturing of cells of the printed bioink.

In some embodiments, the plurality of crosslinkable hydrogel particles are in contact with each other in a container such that interstitial spaces are provided between individual hydrogel particles. The interstitial spaces between the individual particles can form pores in the hydrogel support medium in which a culture medium can be provided and/or flow to the printed bioink during culturing of the cells. The sizes of the pores can be dependent on the sizes of the individual hydrogel particles. For example, smaller pores can result from smaller spaces between the smaller hydrogel particles, and, conversely, larger pores can result from larger spaces between the larger hydrogel particles.

The hydrogel particles can be cytocompatible and, upon degradation, produce substantially non-toxic products. In some embodiments, the hydrogel particles can include a plurality of crosslinkable biodegradable natural or synthetic polymer macromers. The crosslinkable natural polymer macromers can be any crosslinkable hydrogel forming natural polymer or oligomer that includes a functional group (e.g., a carboxylic group) that can be further polymerized, or ionically linked, or interact via hydrophobic/hydrophilic actions, etc. Examples of natural polymers or oligomers are saccharides (e.g., mono-, di-, oligo-, and poly-saccharides), such as glucose, galactose, fructose, lactose and sucrose, collagen, gelatin, glycosaminoglycans, poly(hyaluronic acid), poly(sodium alginate), hyaluronan, alginate, heparin, and agarose. Other examples include polymer macromers, such as chitosan, PEG, PLGA, PCL and other polymers.

The crosslinkable natural polymer macromers can be at least partially crosslinked using any crosslinking means. For example, the crosslinkable natural polymer macromers can be at least partially crosslinked by ionic crosslinking, chemical crosslinking, photocrosslinking or with the aid of click-reactive groups.

In certain embodiments, the crosslinkable natural or synthetic polymer macromer can include dual crosslinkable natural polymer macromers, such as an acrylated and/or methacrylated natural polymer macromers. Acrylated and/or methacrylated natural polymer macromers can include saccharides (e.g., mono-, di-, oligo-, and poly-saccharides), such as glucose, galactose, fructose, lactose and sucrose, collagen, gelatin, glycosaminoglycans, poly(hyaluronic acid), poly(sodium alginate), hyaluronan, alginate, heparin and agarose that can be readily oxidized to form free aldehyde units.

In some embodiments, the acrylated or methacrylated, natural polymer macromers are polysaccharides, which are optionally oxidized so that up to about 50% of the saccharide units therein are converted to aldehyde saccharide units. Control over the degree of oxidation of the natural polymer macromers permits regulation of the gelling time used to form the hydrogel as well as the mechanical properties, which allows for tailoring of the mechanical properties.

In other embodiments, the acrylated and/or methacrylated, natural polymer macromers can include oxidized, acrylated or methacrylated, alginates, which are optionally oxidized so that, for example, up to about 50% of the saccharide units therein are converted to aldehyde saccharide units. Natural source of alginates, for example, from seaweed or bacteria, are useful and can be selected to provide side chains with appropriate M (mannuronate) and G (guluronate) units for the ultimate use of the polymer. Alginate materials can be selected with high guluronate content since the guluronate units, as opposed to the mannuronate units, more readily provide sites for oxidation and crosslinking. Isolation of alginate chains from natural sources can be conducted by conventional methods. See Biomaterials: Novel Materials from Biological Sources, ed. Byrum, Alginates chapter (ed. Sutherland), p. 309-331 (1991). Alternatively, synthetically prepared alginates having a selected M and G unit proportion and distribution prepared by synthetic routes, such as those analogous to methods known in the art, can be used. Further, either natural or synthetic source of alginates may be modified to provide M and G units with a modified structure. The M and/or G units may also be modified, for example, with polyalkylene oxide units of varied molecular weight such as shown for modification of polysaccharides in Spaltro (U.S. Pat. No. 5,490,978) with other alcohols such as glycols. Such modification generally will make the polymer more soluble, which generally will result in a less viscous material. Such modifying groups can also enhance the stability of the polymer. Further, modification to provide alkali resistance, for example, as shown by U.S. Pat. No. 2,536,893, can be conducted.

The oxidation of the natural polymer macromers (e.g., alginate material) can be performed using a periodate oxidation agent, such as sodium periodate, to provide at least some of the saccharide units of the natural polymer macromer with aldehyde groups. The degree of oxidation is controllable by the mole equivalent of oxidation agent, e.g., periodate, to saccharide unit. For example, using sodium periodate in an equivalent % of from 2% to 100%, preferably 1% to 50%, a resulting degree of oxidation, i.e., % if saccharide units converted to aldehyde saccharide units, from about 2% to 50% can be obtained. The aldehyde groups provide functional sites for crosslinking and for bonding tissue, cells, prosthetics, grafts, and other material that is desired to be adhered. Further, oxidation of the natural polymer macromer facilitates their degradation in vivo, even if they are not lowered in molecular weight. Thus, high molecular weight alginates, e.g., of up to 300,000 daltons, may be degradable in vivo, when sufficiently oxidized, i.e., preferably at least 5% of the saccharide units are oxidized.

In some embodiments, the natural polymer macromer (e.g., alginate) can be acrylated or methacrylated by reacting an acryl group or methacryl with a natural polymer or oligomer to form the oxidized, acrylated or methacrylated natural polymer macromer (e.g., alginate). For example, oxidized alginate can be dissolved in a solution chemically functionalized with N-hydroxysuccinimide and 1-ethyl-3-(3-dimethylaminopropyl)-carbodiimide hydrochloride to activate the carboxylic acids of alginate and then reacted with 2-amionethylmethacrylate to provide a plurality of methacrylate groups on the alginate.

The degree of acrylation or methacrylation can be controlled to control the degree of subsequent crosslinking of the acrylate and methacrylates as well as the mechanical properties, and biodegradation rate of the hydrogel particles. The degree of acrylation or methacrylation can be about 1% to about 50%, although this ratio can vary more or less depending on the end use of the composition.

In some embodiments, a solution of natural polymer macromers can be ionically crosslinked and/or chemically crosslinked with a first agent during mixing withing the mixing unit to form a plurality of hydrogel particles. The ionically crosslinked hydrogel can be in the form of a plurality of hydrogel particles. The extent of crosslinking can be controlled by the concentration of CaCl₂. The higher concentration can correspond to a higher extent of crosslinking. The extent of crosslinking alters the mechanical properties of the hydrogel particles and can be controlled as desired for the particular application. In general, a higher degree of crosslinking results in a stiffer gel.

In some embodiments, the hydrogel particles can be crosslinked with a second agent after being printed with the bioink to form dual crosslinked hydrogel particles. A plurality of second crosslink networks can be formed by crosslinking acrylate and/or methacrylate groups of the acrylated or methacrylated natural polymer macromer. The second crosslinking networks formed by crosslinking the acrylate groups or methacrylate groups of the acrylated and/or methacrylated natural polymer macromer can provide improved mechanical properties, such as resistance to excessive swelling, as well as delayed biodegradation rate of the hydrogel particles.

In some embodiments, the acrylate or methacrylate groups of the acrylated and/or methacrylated natural polymer macromer of the hydrogel particles can be crosslinked by photocrosslinking using UV light or visible light in the presence of photoinitiators. For example, acrylated and/or methacrylated natural polymer macromers of the hydrogel particles can be photocrosslinked with a photoinitiator that is provided in the hydrogel support medium. The hydrogel particles can be exposed to a light source at a wavelength and for a time to promote crosslinking of the acrylate groups of the polymers and form the photocrosslinked biodegradable hydrogel particles.

A photoinitiator can include any photo-initiator that can initiate or induce polymerization of the acrylate or methacrylate macromer. Examples of the photoinitiator can include camphorquinone, benzoin methyl ether, 2-hydroxy-2-methyl-1-phenyl-1-propanone, diphenyl(2,4,6-trimethylbenzoyl)phosphine oxide, benzoin ethyl ether, benzophenone, 9,10-anthraquinone, ethyl-4-N,N-dimethylaminobenzoate, diphenyliodonium chloride and derivatives thereof.

In other embodiments, the hydrogel support medium can further include at least one bioactive agent that is provided in the hydrogel particles or potentially a culture medium that can be added to the hydrogel support medium during culturing of the printed bioink. The bioactive agent can include polynucleotides and/or polypeptides encoding or comprising, for example, transcription factors, differentiation factors, growth factors, and combinations thereof. The at least one bioactive agent can also include any agent capable of modulating a function and/or characteristic of a cell and/or promoting tissue formation (e.g., bone and/or cartilage), destruction, and/or targeting a specific disease state (e.g., cancer). Examples of bioactive agents include chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (e.g., EGF), HGF, VEGF, fibroblast growth factors (e.g., bFGF), PDGF, insulin-like growth factor (e.g., IGF-I, IGF-II) and transforming growth factors (e.g., TGF-β I-III), parathyroid hormone, parathyroid hormone related peptide, bone morphogenic proteins (e.g., BMP-2, BMP-4, BMP-6, BMP-7, BMP-12, BMP-13, BMP-14), sonic hedgehog, growth differentiation factors (e.g., GDF5, GDF6, GDF8), recombinant human growth factors (e.g., MP-52 and the MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparin sulfate, polynucleotides, DNA fragments, DNA plasmids, MMPs, TIMPs, interfering RNA molecules, such as siRNAs, miRNAs, DNA encoding for an shRNA of interest, oligonucleotides, proteoglycans, glycoproteins, and glycosaminoglycans.

In some embodiments, a bioactive agent can comprise an interfering RNA or miRNA molecule incorporated on or within insoluble native collagen fibers or dispersed on or within the cell aggregate. The interfering RNA or miRNA molecule can include any RNA molecule that is capable of silencing an mRNA and thereby reducing or inhibiting expression of a polypeptide encoded by the target mRNA. Alternatively, the interfering RNA molecule can include a DNA molecule encoding for a shRNA of interest. For example, the interfering RNA molecule can comprise a short interfering RNA (siRNA) or microRNA molecule capable of silencing a target mRNA that encodes any one or combination of the polypeptides or proteins described above.

### Example

To fabricate alginate microgel slurries, alginate (0.1 g) was dissolved in DMEM (5 ml, 2% w/v) containing 0.05% w/v photoinitiator and then the alginate solution was loaded in a 10-ml syringe. 0.2 ml calcium sulfate slurry (CaSO₄.2H₂O, 0.21 g/ml) was loaded into an another 10-ml syringe. After the two syringes were connected together with a custom-made female-female luer lock mixing device, the two solutions were mixed back and forth 40 times, then further mixed back and forth 10 times every 10 min for 30 min and the alginate slurry was ejected into a printing dish or a cell culture plate.

FIGS. 2(A-B) illustrate images of (A) custom-made female-female luer lock mixing unit and (B) two syringes connected with the mixing unit.

FIG. 3 illustrates microphotographs of OA and OMA microgels made with the mixing unit. Scale bars indicate 500 μm.

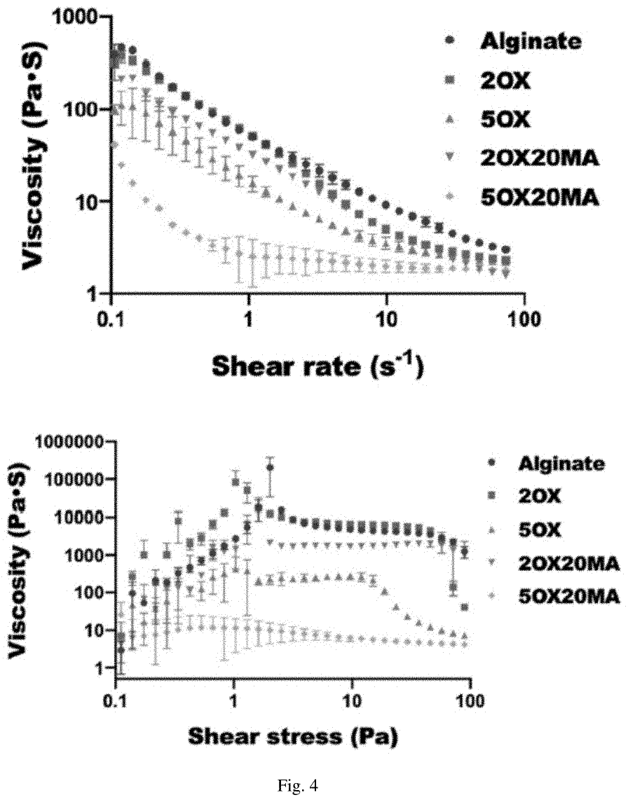

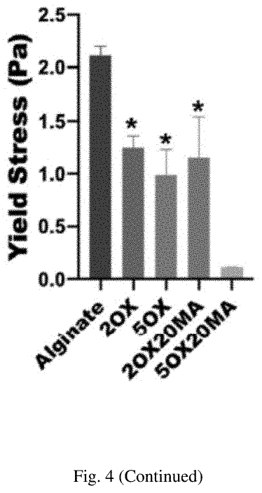

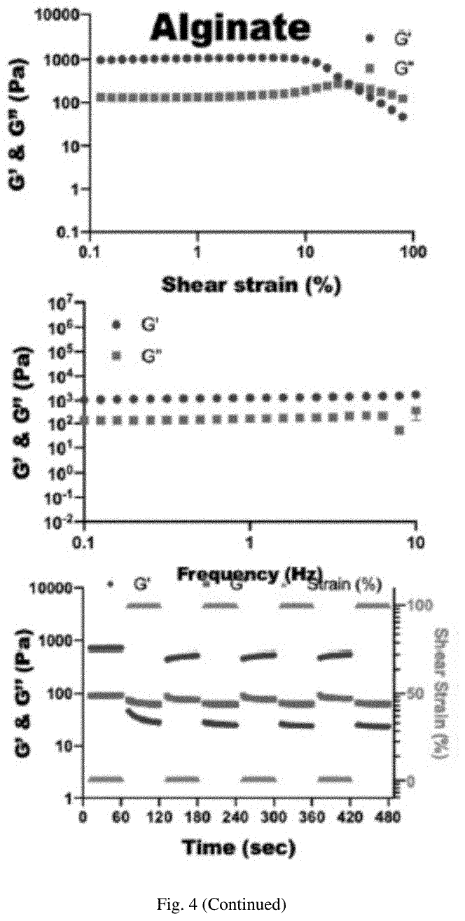

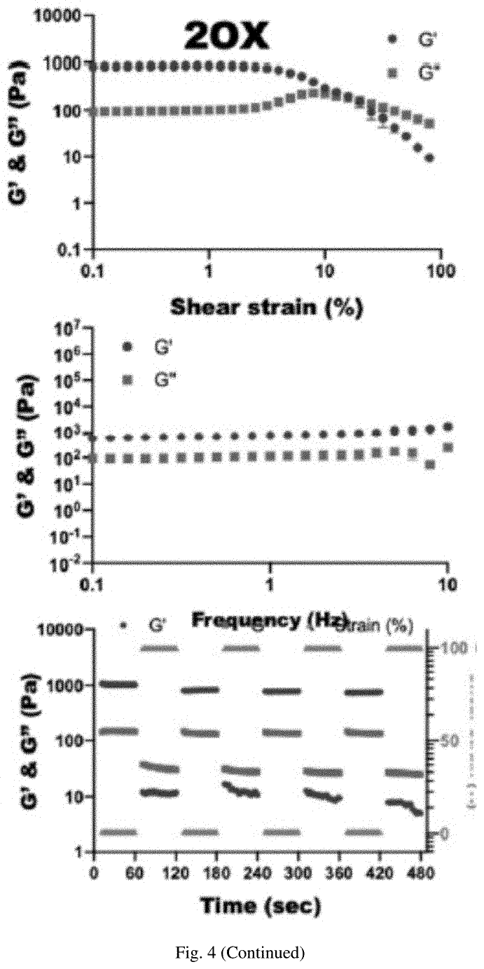

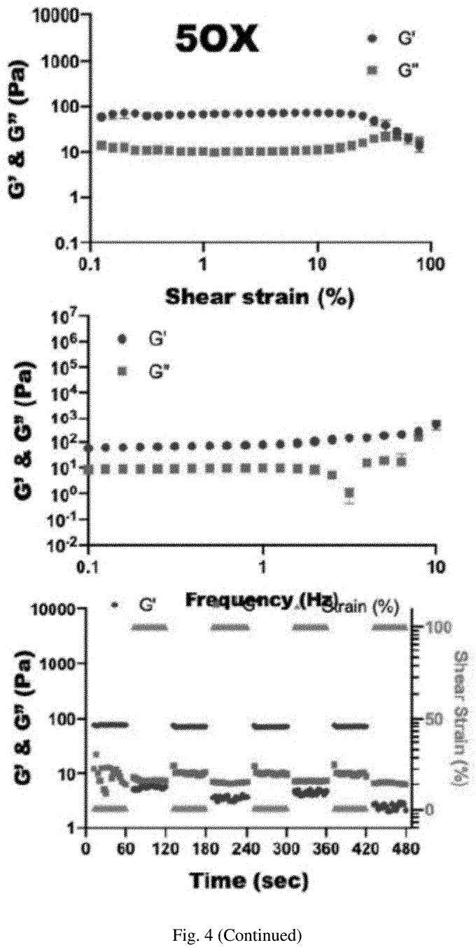

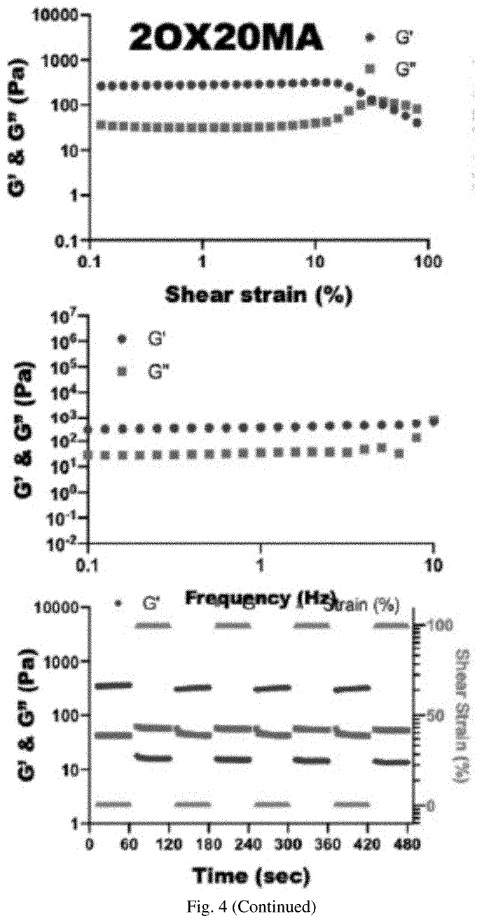

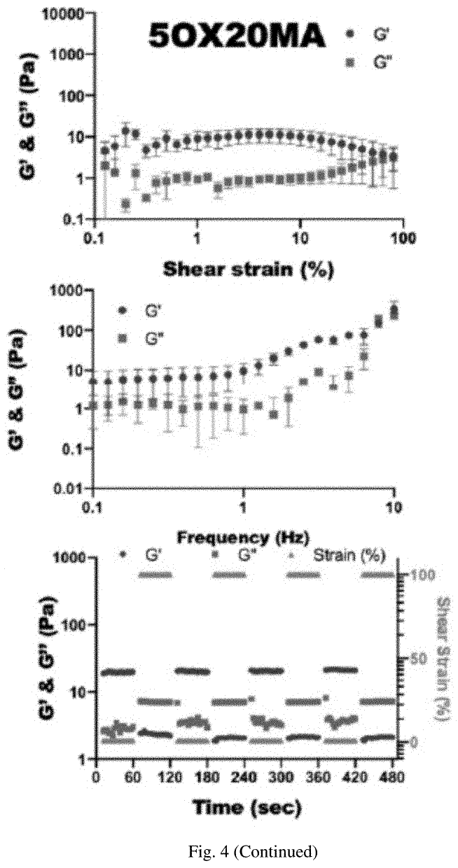

FIG. 4 illustrates plots and graphs showing rheological properties of the alginate slurries formed using the mixing device.

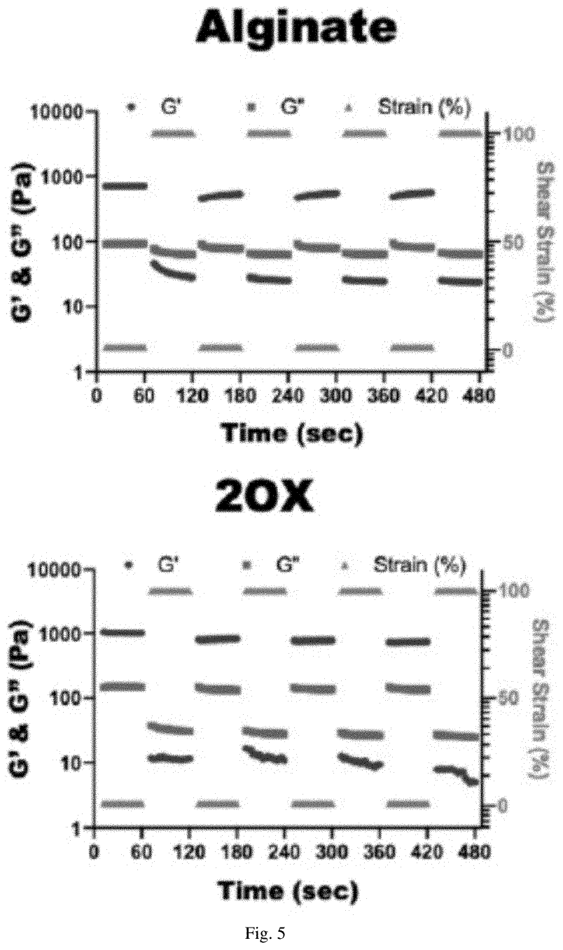

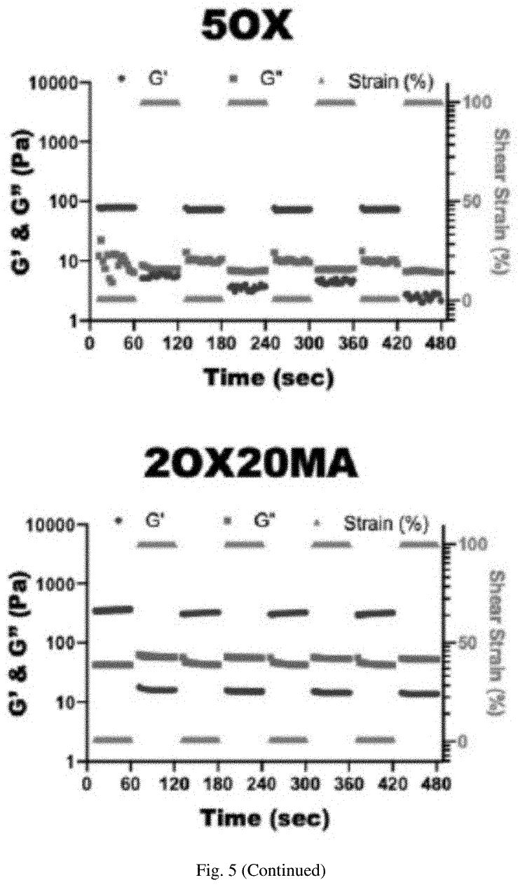

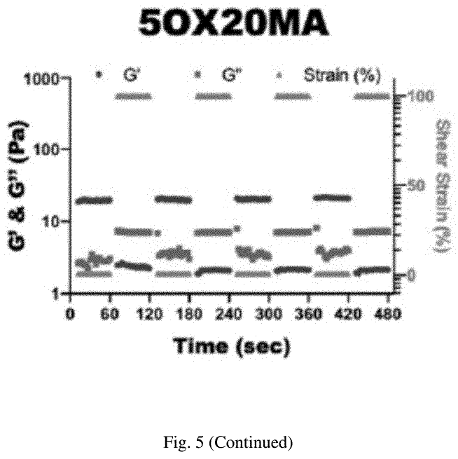

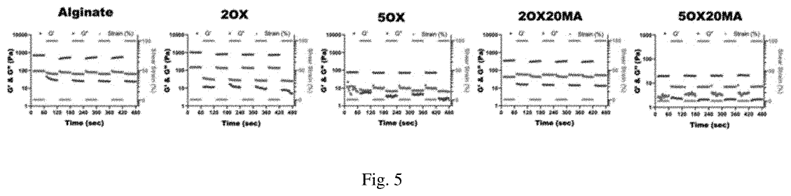

FIG. 5 illustrates plots showing self-healing property of the alginate slurries formed using the mixing device described herein.

FIG. 6 illustrates images and graphs 3D printed hMSC filaments with various needles into the 5OX20MA OMA slurry.

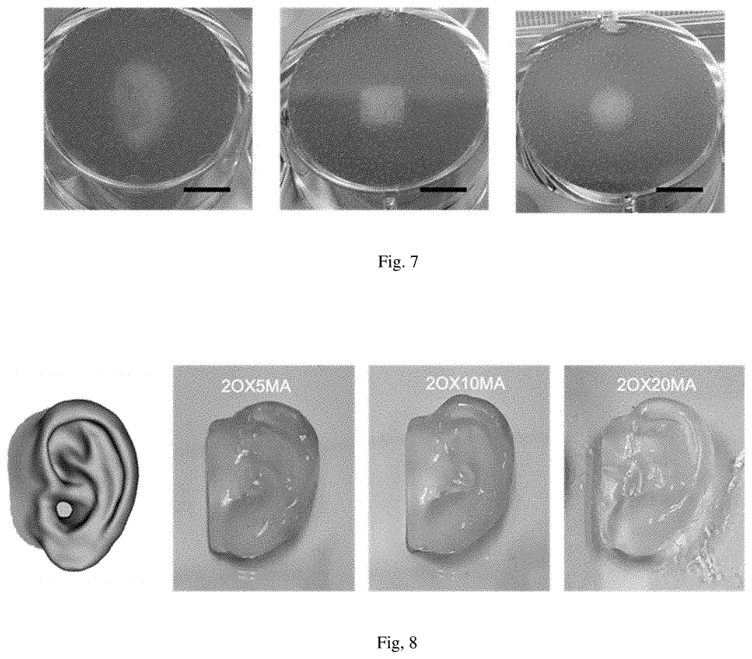

FIG. 7 illustrates 3D printed hMSC-only structures with hMSC into the 5OX20MA OMA slurry.

FIG. 8 illustrates 3D printed ears with various OMA slurries as a bioink using the OMA slurry.

Advantageously, fabrication of alginate slurries is easier: No equipment is needed, and microgel slurries can be made in sterile condition.

No preservatives are needed (e.g., 70% ethanol) for long-term storage: Alginate solution can be stored below −20 C for long-term storage.

No washing process, which causes degradation of the microgel slurries, is needed to remove preservatives.

Alginate with high degree of oxidation can be used for making microgel slurries because alginate slurries formed with higher than 10% oxidation degree using the grinding method degrade during the washing process.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, the following is claimed:

1. A method of forming a gel particle slurry, the method comprising:
providing a first solution that includes a cross-linkable hydrogel polymer macromer and an optional first crosslinker in a first depot and optionally a second solution in a second depot that is separated from the first depot by a mixing unit that includes a mixing element; and
reversibly transferring the first solution and the optional second solution through the mixing unit between the first depot and the second depot such that the first solution and the optional second solution are mixed and agitated to form the gel particle slurry.

2. The method of claim 1, wherein the mixing unit includes a chamber that contains the mixing element.

3. The method of claim 2, wherein the mixing element is a static screw or helical mixing element.

4. The method of claim 1, wherein the mixing element is fixed within the chamber and extends substantially the length of the chamber.

5. The method of claim 5, wherein the first depot and the second depot are connected, respectively, to a first end and second end of the mixing chamber by luer locks.

6. The method of claim 10, wherein the first depot is a first syringe and the second depot is a second syringe.

7. The method of claim 1, wherein the second solution includes the first crosslinker, a second crosslinker, and/or polymer macromer that is capable of crosslinking the cross-linkable hydrogel polymer macromer.

8. The method of claim 1, wherein the first cross-linker is different than the second crosslinker.

9. The method of claim 1, wherein the gel particle slurry includes particles having an average diameter of about 5 nm to about 10 mm.

10. The method of claim 1, wherein the cross-linkable hydrogel polymer macromers are at least partially crosslinked.

11. The method of claim 1, wherein the cross-linkable hydrogel polymer macromer include a plurality of acrylated and/or methacrylated polymer macromers.

12. The method of claim 11, wherein the acrylated and/or methacrylated, polymer macromers are polysaccharides, which are optionally oxidized.

13. The method of claim 1, wherein the second crosslinker is an ionic crosslinker and/or photoinitiator.

14. The method of claim 1, wherein the first cross-linker is an ionic crosslinker and/or photoinitiator.

15. The method of claim 1, wherein cross-linkable hydrogel polymer macromer include oxidized, acrylated and/or methacrylated alginates.

16. The method of claim 1, wherein the first syringe includes an aqueous solution of oxidized acrylated and/or methacrylated alginate and an optional photoinitiator and the second syringe includes a calcium sulfate slurry.

17. The method of claim 1, wherein the first solution and optional second solution are free of preservatives.

18. The method of claim 1, wherein the gel particle slurry is formed without a washing step.

19. The method of claim 1, wherein the chamber, the first depot, and the second depot are sterile.

20. The method of claim 1, wherein the gel particle slurry is self-healing, shear thinning, cross-linkable and/or biocompatible.
